(19) United States
(12) Patent Application Publication
(10) Pub. No.: US 2005/0287105 A1
(43) Pub. Date: Dec. 29, 2005

(54) Solid cosmetic composition textured with an organic copolymer

(75) Inventors: Xavier Blin, Paris (FR); Veronique Ferrari, Maisons-Alfort (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/024,471

(22) Filed: Dec. 30, 2004

Related U.S. Application Data
(63) Continuation-in-part of application No. PCT/EP03/15024, filed on Dec. 8, 2003.
(60) Provisional application No. 60/438,770, filed on Jan. 9, 2003.

(30) Foreign Application Priority Data
Dec. 17, 2002 (FR) 02/16039

(51) Int. Cl. A61K 7/06; A61K 7/11

(52) U.S. Cl. 424/70.122

(57) ABSTRACT

The present invention relates to a solid cosmetic composition characterized in that it comprises a liquid fatty phase textured with an effective amount of at least one organic copolymer and in that it has a hardness ranging from 10 to 250 g and an elasticity of greater than 80%.

This application is a CIP of PCT/EP03/15024 filed on Dec. 8, 2003 under the benefit of a U.S. provisional Application No. 60/438,770 filed on Jan. 9, 2003 and claims the benefits of same applications which are incorporated herein by reference.

The present invention relates to a solid cosmetic composition, especially a makeup and/or care composition, intended to be applied to the skin, the lips and/or the integuments, comprising at least one liquid fatty phase textured with an effective amount of at least one organic copolymer.

It is common to find a structured, i.e. gelled and/or rigidified, liquid fatty phase in cosmetic or dermatological products. This is especially the case in solid compositions, in particular solid cast compositions, lip balms and lipsticks, eyeshadows, concealer products and cast foundations. This structuring is conventionally obtained using waxes or fillers or, more recently, using specific gelling agents.

Documents WO-A-97/36573, U.S. Pat. No. 5,874,069, U.S. Pat. No. 5,919,441, U.S. Pat. No. 6,051,216, WO-A-02/17870, WO-A-02/17871, EP-A-1 177 784, WO-A-99/06473 and U.S. Pat. No. 6,353,076, which is a division of U.S. Pat. No. 6,051,216, propose cosmetic compositions such as deodorant sticks or gels, comprising a silicone oily phase gelled with a polysiloxane/polyamide polymer.

The present invention results more particularly from the observation by the inventors that the use of organic copolymer(s) allows access to a cosmetic composition with a novel solid texture, i.e. a composition with relatively low rigidity and high elasticity. This texture does not correspond either to a stick of the prior art that has relatively high rigidity, or to a gel whose consistency is liquid or pasty.

Consequently, according to a first aspect, the present invention relates to a solid cosmetic composition characterized in that it comprises at least one liquid fatty phase textured with an effective amount of at least one organic copolymer and in that it has a hardness ranging from 10 to 250 g and an elasticity of greater than 80%.

According to another of its aspects, the invention also relates to a solid cosmetic composition characterized in that it comprises at least one dyestuff and a liquid fatty phase textured with an effective amount of at least one organic copolymer and in that it has a hardness ranging from 10 to 250 g and an elasticity of greater than 80%.

The invention also relates to a solid cosmetic composition characterized in that it comprises at least one liquid fatty phase textured with an effective amount of at least two different organic copolymers and in that it has a hardness ranging from 10 to 250 g and an elasticity of greater than 80%.

A subject of the present invention is also a solid cosmetic composition characterized in that it comprises a liquid fatty phase comprising at least one silicone oil, the said fatty phase being textured with an effective amount of at least one organic copolymer and in that it has a hardness ranging from 10 to 250 g and an elasticity of greater than 80%.

According to another of its aspects, the invention similarly relates to a solid cosmetic composition characterized in that it comprises a liquid fatty phase textured with an effective amount of at least one organic copolymer and at least one silicone surfactant and in that it has a hardness ranging from 10 to 250 g and an elasticity of greater than 80%.

More particularly, this copolymer is chosen from:

1) hybrid copolymers comprising at least two units capable of establishing hydrogen interactions, these two units being located in the chain of the copolymer, especially a polyorganosiloxane comprising at least two units capable of establishing hydrogen interactions, these two units being located in the chain of the copolymer, and/or

2) hybrid copolymers comprising at least two units capable of establishing hydrogen interactions, these two units being located on grafts or branches, especially a polyorganosiloxane comprising at least two units capable of establishing hydrogen interactions, these two units being located on grafts or branches.

A subject of the present invention is also a solid cosmetic composition characterized in that it comprises a liquid fatty phase, the said fatty phase being textured with an effective amount of at least two different copolymers of general formula I 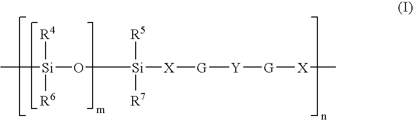
the two copolymers having indices m of different values, one being greater than or equal to 50 and the other less than or equal to 50.

More particularly, a composition as defined above may comprise at least:

1% to 90% by weight of at least one silicone oil,

5% to 50% by weight of at least one copolymer with an index m of greater than or equal to 50, and especially equal to 100,

0.1% to 10% by weight of at least one copolymer with an index m of less than or equal to 50, and especially equal to 15,

0.1% to 10% by weight of a silicone surfactant, and

0.001% to 40% by weight of at least one dyestuff.

A subject of the present invention is also a cosmetic composition for making up the lips, and especially a lipstick, characterized in that it comprises a liquid fatty phase textured with at least one copolymer of general formula I as defined above in which m is greater than or equal to 50.

A subject of the invention is also the use of a copolymer of general formula (I) as defined above, in which m is greater than or equal to 50, in a cosmetic lip makeup composition, for example a lipstick product.

According to another of its aspects, the invention also relates to a solid cosmetic composition characterized in that it comprises at least one liquid fatty phase textured with an effective amount of at least one ethylenic copolymer and in that it has a hardness ranging from 10 to 250 g and an elasticity of greater than 80%.

According to yet another of its aspects, a subject of the present invention is also a process for making up and/or caring for keratin materials, and especially the skin and/or the lips, comprising the application thereto of at least one cosmetic composition in accordance with the present invention.

According to another of its aspects, a subject of the invention is a synthetic support on which is present, on all or part of its surface, at least one coat of a composition according to the invention.

According to another aspect, the invention also relates to a cosmetic assembly comprising:

i) a container delimiting at least one compartment, the said container being closed by means of a closing member; and

ii) a composition placed inside the said compartment, the composition being as defined above.

According to yet another of its aspects, a subject of the invention is the use of at least one organic copolymer, in combination with at least one liquid fatty phase, for the preparation of a cosmetic composition with a hardness ranging from 10 to 250 g and an elasticity of greater than 80%.

Advantageously, the compositions according to the invention have novel textures that may be associated with novel application behaviour, which is especially the case for products such as lipsticks in which the product may be applied to the lips directly, without using an applicator.

More specifically, the compositions according to the invention are not in a hard solid form like a stick or in a fluid form like a gloss, or even in a form of soft paste type, but rather in the form of a mousse or gum texture simultaneously having soft and elastic properties.

The soft and elastic solid textures obtained according to the invention are compatible with direct application to the lips without using an applicator, as in the case of fluid textures. They provide suppleness, softness and very good elasticity on application, thus preserving the product for a future application. The product does not become permanently deformed, but rather regains its initial form after application.

Advantageously, the composition in accordance with the invention, in the form of a stick, has the behaviour of a deformable and soft elastic solid, giving noteworthy elastic softness on application.

For the purposes of the patent application, the term “liquid fatty phase” means a fatty phase that is liquid at room temperature (25° C.) and atmospheric pressure (760 mmHg), composed of one or more mutually compatible fatty substances that are liquid at room temperature, also known as oils.

The term “textured liquid fatty phase” means a fatty phase whose viscosity is increased by adding the organic copolymer.

The term “solid composition” denotes a composition that does not flow under its own weight. This capacity not to flow under its own weight may in particular be determined by placing the composition in a transparent glass container after having delimited beforehand the area on which the sample is deposited. The sample is thus left for about 12 hours at a temperature of 25° C. After this period, no flow beyond the deposition area is observed with the naked eye.

The compositions according to the invention advantageously have a hardness ranging from 10 to 250 g and an elasticity of greater than 80%.

Measurement of the Elasticity and the Hardness

The hardness and the elasticity of the composition according to the invention may be measured using a texturometer, which makes it possible to obtain the variation in the resistance to deformation of the composition as a function of the displacement of a spindle into a sample of the said composition.

The texturometer measures the force of resistance to deformation of the composition as soon as the spindle comes into contact with the sample. After having reached a maximum programmed depth L0 into the sample, the spindle returns to the initial point.

The hardness (expressed in grams or in newtons) is equal to the value of the resistance of the composition when the spindle is at the end of its course, and the elasticity (expressed as a percentage) is equal to the ratio of i) the distance L at which contact between the spindle and the sample is broken during the withdrawal of the spindle, and of ii) the distance L0. The breaking of contact is reflected by the disappearance of the force of resistance of the composition on the spindle. The elasticity is proportional to the distance to which the system “accompanies” the rise of the spindle; the larger its value, the more elastic the system.

The texturometer used may especially be a Stable Micro System TAX-T2i ® texturometer equipped with the Texture Expert Exceed® operating software and fitted with a P/0.5 HS Rheo hemispheric plastic spindle.

The parameters applied are advantageously the following:

speed before contact: 0.1 mm.s⁻¹,

speed of displacement into the sample: 0.1 mm.s⁻¹,
speed of withdrawal: 0.1 mm.s⁻¹,
maximum depth L0: 1 mm.

The samples of composition are prepared by hot-casting a sufficient amount of the test composition, for example into a 100×5 mm pretared Petri dish, to obtain a sample about 1 cm thick. The advantage of this choice of packaging is its width, which is sufficient to overcome any edge effect. Two Petri dishes are thus prepared and are left to stand for a minimum of 24 hours at 20° C. before characterization.

At least three measurements are taken on each sample: one, at the centre, and others, at points equidistant from the centre and from the edge of the Petri dish.

The hardness and the elasticity are equal to the mean of a minimum number of six measurements taken.

The hardness of the composition according to the invention is such that the composition is self-supporting and can readily be broken down to form a satisfactory deposit on the skin and the lips. In addition, with this hardness, the composition of the invention has good impact strength.

The compositions according to the invention advantageously have an elasticity of greater than 90% and in particular close to 100%.

More particularly, the rigidity may range from 30 g to 200 g, especially from 50 g to 175 g and more particularly from 75 g to 110 g. As mentioned previously, these physical properties of the compositions according to the invention are obtained by virtue of the texturing of their liquid fatty phase with an effective amount of at least one organic copolymer, also known as a “texturing copolymer”, according to the invention.

The texturing copolymers may be chosen in terms of nature and amount, according to the desired texture or hardness and the desired stability of the compositions and as a function of the intended specific application.

The amount of the (at least one) texturing copolymer may be such that it leads to the production of a disintegrable solid that does not flow under the effect of its own weight.

Organic Copolymer

For the purposes of the invention, the term “copolymer” means a compound obtained by reaction of two chemical compounds of different structure, at least one of which is an organic compound. This copolymer may be a hybrid copolymer obtained by reacting a hydrocarbon-based monomer and a silicone monomer or alternatively an ethylenic copolymer.

More particularly, this hybrid copolymer may be chosen from:

1) hybrid copolymers comprising at least two units capable of establishing hydrogen interactions, these two units being located in the chain of the copolymer, especially a polyorganosiloxane comprising at least two units capable of establishing hydrogen interactions, these two units being located in the chain of the copolymer, and/or

2) hybrid copolymers comprising at least two units capable of establishing hydrogen interactions, these two units being located on grafts or branches, especially a polyorganosiloxane comprising at least two units capable of establishing hydrogen interactions, these two units being located on grafts or branches.

Representatives of compounds of the family 1) that may especially be mentioned include the compounds of formula I more particularly described below:

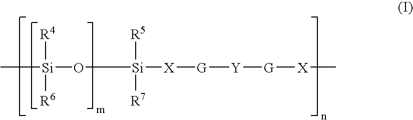

As regards the compounds of the family 2), they may especially be represented by the compounds of formula (II) more particularly described below.

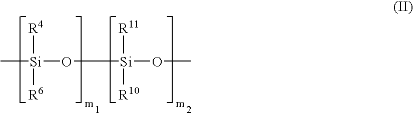

As regards the units capable of establishing hydrogen bonding, they may be chosen from ester, amide, sulfonamide, carbamate, thiocarbamate, urea, urethane, thiourea, oxamido, guanidino and biguanidino, and combinations thereof.

In the composition of the invention, the organic copolymer generally represents from 0.5% to 80%, especially from 2% to 60%, in particular from 5% to 40% and more particularly from 3% to 30% by weight relative to the total weight of the composition.

Moreover, the texturing copolymer/liquid fatty phase mass ratio of the composition is preferably from 0.01 to 10, more preferably from 0.05 to 5 and better still from 0.1 to 2.

Hybrid Copolymer

According to a first variant, the copolymers used as texturing agents in the composition of the invention may be polymers of the polyorganosiloxane type such as those described in documents U.S. Pat. No. 5,874,069, U.S. Pat. No. 5,919,441, U.S. Pat. No. 6,051,216 and U.S. Pat. No. 5,981,680.

a) According to a first variant, they are polyorganosiloxanes as defined above and of which the units capable of establishing hydrogen interactions are located in the chain of the copolymer.

The polymers may more particularly be polymers comprising at least one unit corresponding to the general formula I: 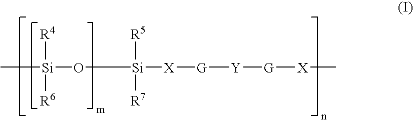
in which:

1) R⁴₁, R⁵, R⁶ and R⁷, which may be identical or different, represent a group chosen from:

linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,

C₆ to C₁₀ aryl groups, optionally substituted with one or more C₁ to C₄ alkyl groups,

polyorganosiloxane chains possibly containing one or more oxygen, sulfur and/or nitrogen atoms;

2) the groups X, which may be identical or different, represent a linear or branched C₁ to C₃₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups, or

4) Y represents a group corresponding to the formula: 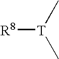
in which

T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer;

5) the groups G, which may be identical or different, represent divalent groups chosen from: 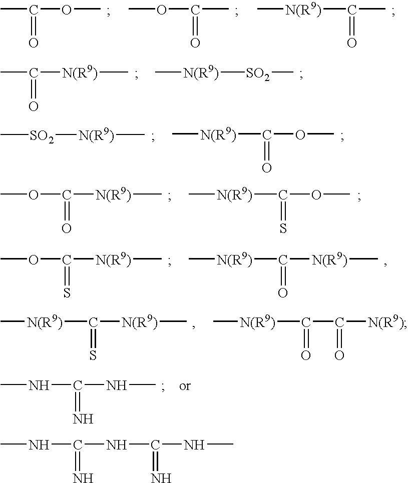
in which R⁹ represents a hydrogen atom or a linear or branched C₁ to C₂₀ alkyl group, on condition that at least 50% of the groups R⁹ of the polymer represent a hydrogen atom and that at least two of the groups G of the polymer are a group other than:

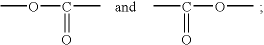

6) n is an integer ranging from 2 to 500 and preferably from 2 to 200, and m is an integer ranging from 1 to 1000, preferably from 1 to 700 and better still from 6 to 200.

According to the invention, 80% of the groups R⁴, R⁵, R⁶ and R⁷ of the polymer are preferably chosen from methyl, ethyl, phenyl and 3,3,3-trifluoropropyl groups.

According to the invention, Y can represent various divalent groups, furthermore optionally comprising one or two free valencies to establish bonds with other units of the polymer or copolymer. Preferably, Y represents a group chosen from:

a) linear C₁ to C₂₀ and preferably C₁ to C₁₀ alkylene groups,

b) C₃₀ to C₅₆ branched alkylene groups possibly comprising rings and non-conjugated unsaturations,

c) C₅-C₆ cycloalkylene groups,

d) phenylene groups optionally substituted with one or more C₁ to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cyclo-alkane, C₁ to C₃ hydroxyalkyl and C₁ to C₆ alkylamine groups,

g) polyorganosiloxane chains of formula: 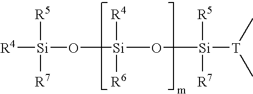
in which R⁴, R⁵, R⁶, R⁷, T and m are as defined above, and

h) polyorganosiloxane chains of formula:

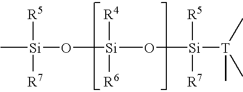

b) According to the second variant, the polyorganosiloxanes may be polymers comprising at least one unit corresponding to formula (II): 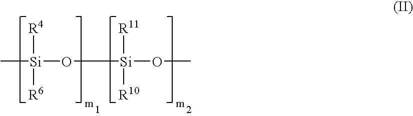
in which

R⁴ and R⁶, which may be identical or different, are as defined above for formula (I),

R¹⁰ represents a group as defined above for R⁴ and R⁶₁ or represents a group of formula —X-G-R¹² in which X and G are as defined above for formula (I) and R¹² represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,

R¹¹ represents a group of formula —X-G-R¹² in which X, G and R¹² are as defined above,

m₁ is an integer ranging from 1 to 998, and

m₂ is an integer ranging from 2 to 500.

According to the invention, the polymer used as structuring agent may be a homopolymer, i.e. a polymer comprising several identical units, in particular units of formula (I) or of formula (II).

According to the invention, it is also possible to use a polymer consisting of a copolymer comprising several different units of formula (I), i.e. a polymer in which at least one of the groups R⁴, R⁵, R⁶, R⁷, X, G, Y, m and n is different in one of the units. The copolymer may also be formed from several units of formula (II), in which at least one of the groups R⁴, R⁶₁ R¹⁰, R¹¹, m₁ and m₂ is different in at least one of the units.

It is also possible to use a copolymer comprising at least one unit of formula (I) and at least one unit of formula (II), the units of formula (I) and the units of formula (II) possibly being identical to or different from each other.

According to one variant of the invention, it is also possible to use a copolymer furthermore comprising at least one hydrocarbon-based unit comprising two groups capable of establishing hydrogen interactions, chosen from ester, amide, sulfonamide, carbamate, thiocarbamate, urea, urethane, thiourea, oxamido, guanidino and biguanidino groups, and combinations thereof.

These copolymers may be block copolymers or grafted copolymers.

1) According to a first embodiment of the invention, the groups capable of establishing hydrogen interactions are amide groups of formulae —C(O)NH— and —HN—C(O)—.

In this case, the structuring agent may be a polymer comprising at least one unit of formula (III) or (IV): 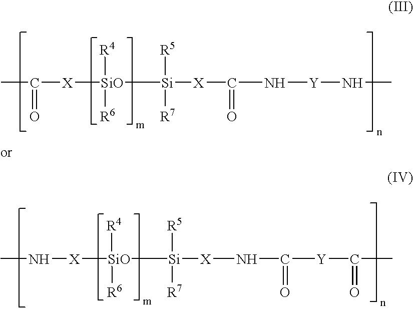
in which R⁴, R⁵₁ R⁶, R⁷, X, Y, m and n are as defined above.

Such a unit may be obtained:

either by a condensation reaction between a silicone containing α,ω-carboxylic acid ends and one or more diamines, according to the following reaction scheme:

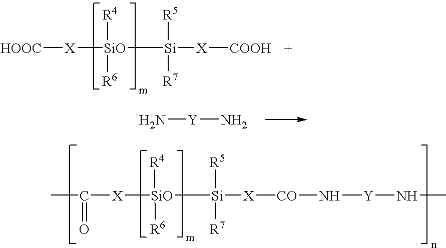

or by reaction of two molecules of α-unsaturated carboxylic acid with a diamine according to the following reaction scheme: 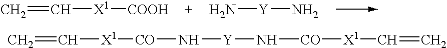
followed by the addition of a siloxane to the ethylenic unsaturations, according to the following scheme: 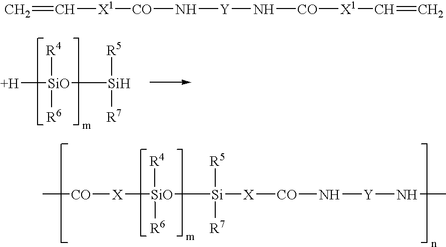
in which X¹—(CH₂)₂— corresponds to X defined above and Y, R⁴, R⁵, R⁶, R⁷ and m are as defined above;

or by reaction of a silicone containing α,ω-NH₂ ends and a diacid of formula HOOC—Y—COOH according to the following reaction scheme:

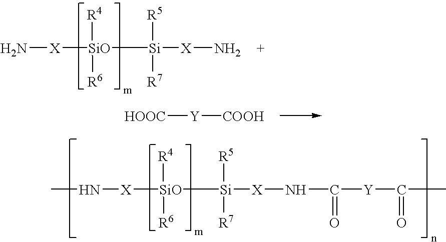

In these polyamides of formula (III) or (IV), m is preferably in the range from 1 to 700, in particular from 15 to 500 and especially from 50 to 200. As regards n, it is in particular in the range from 1 to 500, preferably from 1 to 100 and better still from 4 to 25,

X is preferably a linear or branched alkylene chain containing from 1 to 30 carbon atoms, in particular 1 to 20 carbon atoms, especially from 5 to 15 carbon atoms and more particularly 10 carbon atoms, and

Y is preferably an alkylene chain that is linear or branched or that possibly comprises rings and/or unsaturations, containing from 1 to 40 carbon atoms, in particular from 1 to 20 carbon atoms and better still from 2 to 6 carbon atoms, in particular 6 carbon atoms.

In formulae (III) and (IV), the alkylene group representing X or Y can optionally contain in its alkylene portion at least one of the following elements:

1) 1 to 5 amide, urea, urethane or carbamate groups,

2) a C₅ or C₆ cycloalkyl group, and

3) a phenylene group optionally substituted with 1 to 3 identical or different C₁ to C₃ alkyl groups.

In formulae (III) and (IV), the alkylene groups may also be substituted with at least one element chosen from the group consisting of:

a hydroxyl group,

a C₃ to C₈ cycloalkyl group,

one to three C₁ to C₄₀ alkyl groups,

a phenyl group optionally substituted with one to three C₁ to C₃ alkyl groups,

a C₁ to C₃ hydroxyalkyl group, and

a C₁ to C₆ aminoalkyl group.

In these formulae (III) and (IV), Y may also represent: 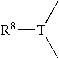
in which R⁸ represents a polyorganosiloxane chain and T represents a group of formula: 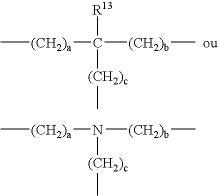
in which a, b and c are, independently, integers ranging from 1 to 10, and R¹³ is a hydrogen atom or a group such as those defined for R⁴, R⁵, R⁶ and R⁷.

In formulae (III) and (IV), R ⁴ R⁵, R⁶ and R⁷ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different units of formula (III) or (IV).

Thus, the polymer may be a polyamide containing several units of formula (III) or (IV) of different lengths, i.e. a polyamide corresponding to formula (V): 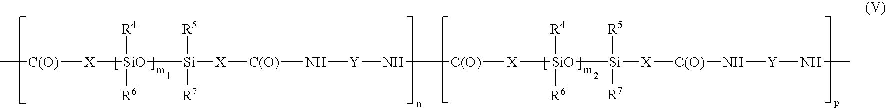
in which X, Y, n and R⁴ to R⁷ have the meanings given above, m₁ and m₂, which are different, are chosen in the range from 1 to 1000, and p is an integer ranging from 2 to 300.

In this formula, the units may be structured to form either a block copolymer, or a random copolymer or an alternating copolymer. In this copolymer, the units may be not only of different lengths, but also of different chemical structures, for example containing different groups Y. In this case, the copolymer may correspond to formula VI: 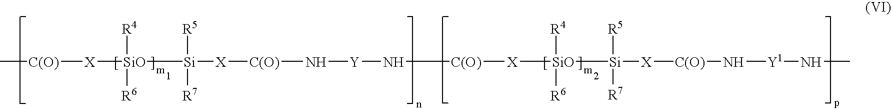
in which R⁴ to R⁷, X, Y, m₁, m₂, n and p have the meanings given above and Y¹ is different from Y but chosen from the groups defined for Y. As previously, the various units may be structured to form either a block copolymer, or a random copolymer or an alternating copolymer.

In this first embodiment of the invention, the structuring agent may also consist of a grafted copolymer. Thus, the polyamide containing silicone units may be grafted and optionally crosslinked with silicone chains containing amide groups. Such polymers may be synthesized with trifunctional amines.

In this case, the copolymer may comprise at least one unit of formula (VII): 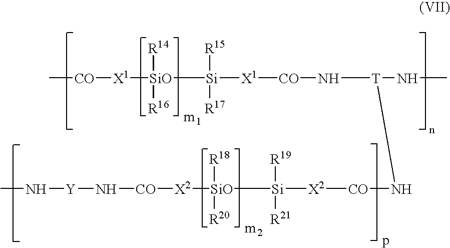
in which X¹ and X², which may be identical or different, have the meaning given for X in formula (I), n is as defined in formula (I), Y and T are as defined in formula (I), R¹⁴ to R²¹ are groups chosen from the same group as R⁴ to R⁷, m₁ and m₂ are numbers in the range from 1 to 1000, and p is an integer ranging from 2 to 500.

In formula (VII), it is preferred that:

p is in the range from 1 to 25 and better still from 1 to 7,

R¹⁴ to R²¹ are methyl groups,

T corresponds to one of the following formulae: 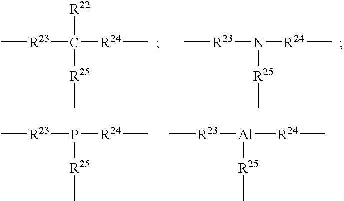
in which R²2 is a hydrogen atom or a group chosen from the groups defined for R⁴ to R⁷, and R²³ , R²⁴ and R²⁵ are, independently, linear or branched alkylene groups, and more preferably correspond to the formula: 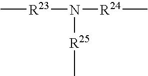
in particular with R²³₁ R²⁴ and R²⁵ representing —CH₂—CH₂—,

m₁ and m₂ are in the range from 15 to 500 and better still from 15 to 45,

X¹ and X² represent —(CH₂)₁₀—, and

Y represents —CH₂—.

These polyamides containing a grafted silicone unit of formula (VII) may be copolymerized with polyamide-silicones of formula (II) to form block copolymers, alternating copolymers or random copolymers. The weight percentage of grafted silicone units (VII) in the copolymer may range from 0.5% to 30% by weight.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to the invention, the preferred siloxane-based polyamides are:

polyamides of formula (III) in which m ranges from 50 to 600, in particular from 60 to 400 and especially from 75 to 200, and is more particularly about 100;

mixtures of polyamide of formula (III) combining:

1) 80% to 99% by weight of a polyamide in which m ranges from 50 to 600, in particular from 60 to 400 and especially from 75 to 200, and is more particularly about 100, and

2) 1% to 20% of a polyamide in which m is in the range from 5 to 100 and in particular from 10 to 75, and is more particularly about 15;

mixtures of polyamide of formula (III) combining:

1) 80% to 99% by weight of a polyamide in which n ranges from 2 to 10 and in particular from 3 to 6, and

2) 1% to 20% of a polyamide in which n is in the range from 5 to 500 and in particular from 30 to 100;

mixtures of polyamide of formula (III) combining:

1) 1% to 20% by weight of a polyamide in which n is equal to 2 to 10 and in particular from 3 to 6, and

2) 80% to 99% by weight of a polyamide in which n is in the range from 30 to 500 and in particular from 30 to 100;

polyamides of formula (III) in which X represents C₃ to C₁₅, in particular C₅ to C₁₂ and especially C₁₀ alkyl radical, and

polyamides of formula (III) in which Y represents a C₃ to C₁₀, in particular C₄ to C₈ and especially C₆ alkyl radical.

According to one embodiment variant of the invention, it is possible to use a copolymer of silicone polyamide and of hydrocarbon-based polyamide, i.e. a copolymer comprising units of formula (III) or (IV) and hydrocarbon-based polyamide units. In this case, the polyamide-silicone units may be located at the ends of the hydrocarbon-based polyamide.

The composition according to the invention advantageously comprises at least one polydimethylsiloxane block copolymer of general formula (I) with an index m of greater than 50, in particular greater than 75 and especially of about 100.

According to one particular embodiment, such a composition also comprises at least one polydimethylsiloxane block copolymer of general formula (I) with an index m of 15.

Polyamide-based texturing copolymers containing silicones may be produced by silylic amidation of polyamides based on fatty acid dimer. This approach involves the reaction of free acid sites existing on a polyamide as end sites, with organosiloxane-monoamines and/or organosiloxane-diamines (amidation reaction), or alternatively with oligosiloxane alcohols or oligosiloxane diols (esterification reaction). The esterification reaction requires the presence of acid catalysts, as is known in the art. It is desirable for the polyamide containing free acid sites, used for the amidation or esterification reaction, to have a relatively high number of acid end groups (for example polyamides with high acid numbers, for example from 15 to 20).

For the amidation of the free acid sites of the hydrocarbon-based polyamides, siloxane diamines with 1 to 300, more particularly 2 to 50 and better still 2, 6, 9.5, 12, 13.5, 23 or 31 siloxane groups, may be used for the reaction with hydrocarbon-based polyamides based on fatty acid dimers.

The reactions may be carried out in xylene to extract the water produced from the solution by azeotropic distillation, or at higher temperatures (about 180 to 200° C.) without solvent. Typically, the efficacy of the amidation and the reaction rates decrease when the siloxane diamine is longer, i.e. when the number of siloxane groups is higher. Free amine sites may be blocked after the initial amidation reaction of the diaminosiloxanes by reacting them either with a siloxane acid, or with an organic acid such as benzoic acid.

For the esterification of the free acid sites on the polyamides, this may be performed in boiling xylene with about 1% by weight, relative to the total weight of the reagents, of para-toluenesulfonic acid as catalyst.

These reactions carried out on the carboxylic acid end groups of the polyamide lead to the incorporation of silicone units only at the ends of the polymer chain.

It is also possible to prepare a copolymer of polyamide-silicone, using a polyamide containing free amine groups, by amidation reaction with a siloxane containing an acid group.

It is also possible to prepare a structuring agent based on a copolymer between a hydrocarbon-based polyamide and a silicone polyamide, by transamidation of a polyamide having, for example, an ethylenediamine constituent, with an oligosiloxane-a,o-diamine, at high temperature (for example 200 to 3000C), to carry out a transamidation such that the ethylenediamine component of the original polyamide is replaced with the oligosiloxane diamine.

The copolymer of hydrocarbon-based polyamide and of polyamide-silicone may also be a grafted copolymer comprising a hydrocarbon-based polyamide backbone with pendent oligosiloxane groups.

This may be obtained, for example:

by hydrosilylation of unsaturated bonds in polyamides based on fatty acid dimers;

by silylation of the amide groups of a polyamide; or

by silylation of unsaturated polyamides by means of an oxidation, i.e. by oxidizing the unsaturated groups into alcohols or diols, to form hydroxyl groups that are reacted with siloxane carboxylic acids or siloxane alcohols. The olefinic sites of the unsaturated polyamides may also be epoxidized and the epoxy groups may then be reacted with siloxane amines or siloxane alcohols.

2) According to a second embodiment of the invention, the texturing copolymer consists of a homopolymer or a copolymer comprising urethane or urea groups.

As previously, the polymer may comprise polyorganosiloxane units containing two or more urethane and/or urea groups, either in the backbone of the polymer or on side chains or as pendent groups.

The polymers comprising at least two urethane and/or urea groups in the backbone may be polymers comprising at least one unit corresponding to the following formula (VIII): 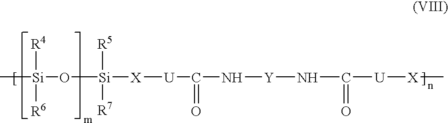
in which R⁴, R⁵, R⁶, R⁷, X, Y, m and n have the meanings given above for formula (I), and U represents —O— or —NH—, such that: 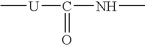
corresponds to a urethane or urea group.

In this formula (VIII), Y may be a linear or branched C₁ to C₄₀ alkylene group, optionally substituted with a C₁ to C₁₅ alkyl group or a C₅ to C₁₀ aryl group. Preferably, a —(CH₂)₆— group is used.

Y may also represent a C₅ to C₁₂ cycloaliphatic or aromatic group that may be substituted with a C₁ to C₁₅ alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane. Generally, it is preferred for Y to represent a linear or branched C₁ to C₄₀ alkylene radical or a C₄ to C₁₂ cycloalkylene radical.

Y may also represent a polyurethane or polyurea block corresponding to the condensation of several diisocyanate molecules with one or more molecules of coupling agents of the diol or diamine type. In this case, Y comprises several urethane or urea groups in the alkylene chain.

It may correspond to formula (IX): 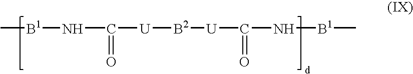
in which B¹ is a group chosen from the groups given above for Y, U is —O— or —NH— and B² is chosen from:
linear or branched C₁ to C₄₀ alkylene groups,
C₅ to C₁₂ cycloalkylene groups, optionally bearing alkyl substituents, for example one to three methyl or ethyl groups, or alkylene, for example the diol radical: cyclohexanedimethanol,
phenylene groups that may optionally bear C₁ to C₃ alkyl substituents, and
groups of formula: 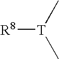
   in which T is a hydrocarbon-based trivalent radical possibly containing one or more hetero atoms such as oxygen, sulfur and nitrogen and R⁸ is a polyorgano-siloxane chain or a linear or branched C₁ to C₅₀ alkyl chain.

T can represent, for example:

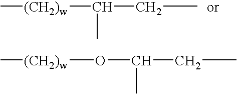

with w being an integer ranging from 1 to 10 and R⁸ being a polyorganosiloxane chain.

When Y is a linear or branched C₁ to C₄₀ alkylene group, the —(CH₂)₂— and —(CH₂)₆— groups are preferred.

In the formula given above for Y, d may be an integer ranging from 0 to 5, preferably from 0 to 3 and more preferably equal to 1 or 2.

Preferably, B² is a linear or branched C₁ to C₄₀ alkylene group, in particular —(CH₂)₂— or —(CH₂)₆— or a group: 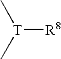
with R⁸ being a polyorganosiloxane chain.

As previously, the polymer constituting the texturing copolymer may be formed from silicone urethane and/or silicone urea units of different length and/or constitution, and may be in the form of block or random copolymers.

The polymers of formula (VIII) comprising urea or urethane groups in the chain of the silicone polymer may be obtained by reaction between a silicone containing α,ω—NH₂ or —OH end groups, of formula: 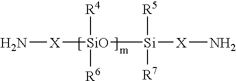
in which m, R⁴, R⁵, R⁶₁ R⁷ and X are as defined for formula (I), and a diisocyanate OCN—Y—NCO in which Y has the meaning given in formula (I); and optionally a diol or diamine coupling agent of formula H₂N—B²—NH₂ or HO—B²—OH, in which B² is as defined in formula (IX).

According to the stoichiometric proportions between the two reagents, diisocyanate and coupling agent, Y may have the formula (IX) with d equal to 0 or d equal to 1 to 5.

As in the case of the polyamide silicones of formula (IV), (II) or (III), it is possible to use in the invention polyurethane or polyurea silicones containing units of different length and structure, in particular units whose lengths differ by the number of silicone units. In this case, the copolymer may correspond, for example, to the formula: 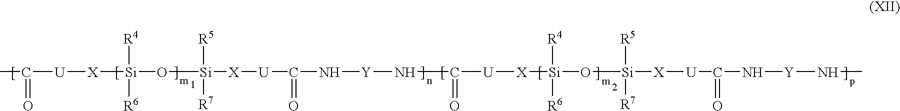
in which R⁴, R⁵, R⁶, R⁷, X, Y and U are as defined for formula (VIII) and m₁, m₂, n and p are as defined for formula (V).

According to the invention, the silicone may also comprise urethane and/or urea groups no longer in the backbone but as side branches.

In this case, the polymer may comprise at least one unit of formula: 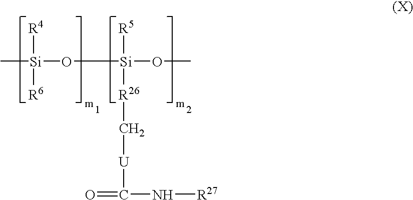
in which R⁴, R⁶, R , m₁ and m₂ have the meanings given above for formula (II), and R⁵ for formula (I),

U represents C or NH,

R²⁶ represents a C₁ to C₄₀ alkylene group, optionally comprising one or more hetero atoms chosen from O and N, or a phenylene group, and

R²⁷ is chosen from linear, branched or cyclic, saturated or unsaturated C₁ to C₅₀ alkyl groups, and phenyl groups optionally substituted with one to three C₁ to C₃ alkyl groups.

The polymers comprising at least one unit of formula (X) contain siloxane units and urea or urethane groups, and they may be used as texturing copolymers in the compositions of the invention.

The siloxane polymers may have a single urea or urethane group by branching or may have branches containing two urea or urethane groups, or alternatively they may contain a mixture of branches containing one urea or urethane group and branches containing two urea or urethane groups.

They may be obtained from branched polysiloxanes, comprising one or two amino groups per branch, by reacting these polysiloxanes with monoisocyanates.

As examples of starting polymers of this type containing amino and diamino branches, mention may be made of the polymers corresponding to the following formulae:

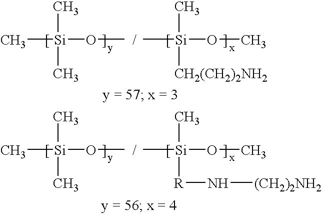

In these formulae, the symbol “/” indicates that the segments may be of different lengths and in a random order, and R represents a linear aliphatic group preferably containing 1 to 6 carbon atoms and better still 1 to 3 carbon atoms.

Such polymers containing branching may be formed by reacting a siloxane polymer, containing at least three amino groups per polymer molecule, with a compound containing only one monofunctional group (for example an acid, an isocyanate or an isothiocyanate) to react this monofunctional group with one of the amino groups and to form groups capable of establishing hydrogen interactions. The amino groups may be on side chains extending from the main chain of the siloxane polymer, such that the groups capable of establishing hydrogen interactions are formed on these side chains, or alternatively the amino groups may be at the ends of the main chain, such that the groups capable of hydrogen interaction will be end groups of the polymer.

As a procedure for forming a polymer containing siloxane units and groups capable of establishing hydrogen interactions, mention may be made of the reaction of a siloxane diamine and of a diisocyanate in a silicone solvent so as to provide a gel directly. The reaction may be performed in a silicone fluid, the resulting product being dissolved in the silicone fluid, at high temperature, the temperature of the system then being reduced to form the gel.

The polymers that are preferred for incorporation into the compositions according to the present invention are siloxane-urea copolymers that are linear and that contain urea groups as groups capable of establishing hydrogen interactions in the backbone of the polymer.

As an illustration of a polysiloxane ending with four urea groups, mention may be made of the polymer of formula: 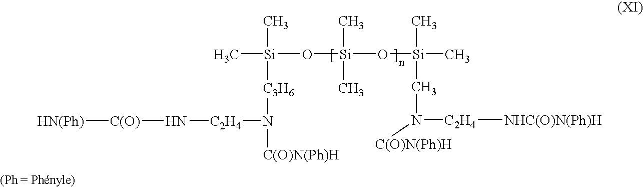
in which Ph is a phenyl group and n is a number from 0 to 300 and in particular from 0 to 100, for example 50.

This polymer is obtained by reacting the following polysiloxane containing amino groups: 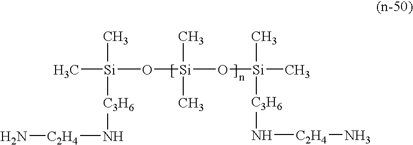
with phenyl isocyanate.

Branched polyurethane or polyurea silicones may also be obtained by using, instead of the diisocyanate OCN—Y—NCO, a triisocyanate of formula:

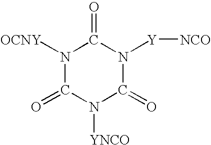

A polyurethane or polyurea silicone containing branches comprising an organosiloxane chain with groups capable of establishing hydrogen interactions is thus obtained. Such a polymer comprises, for example, a unit corresponding to the formula: 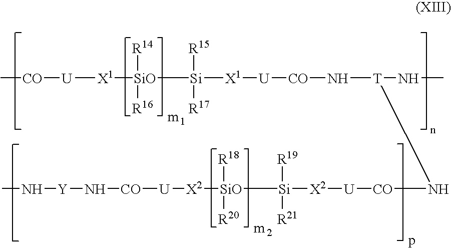
in which X¹ and X², which are identical or different, have the meaning given for X in formula (I), n is as defined in formula (I), Y and T are as defined in formula (I), R¹⁴ to R²¹ are groups chosen from the same group as R⁴ to R⁷, m₁ and m₂ are numbers in the range from 1 to 1000, and p is an integer ranging from 2 to 500.

As in the case of the polyamides, this copolymer can also comprise polyurethane silicone units without branches.

The siloxane-based polyureas and polyurethanes that are preferred are:

polymers of formula (VIII) in which m ranges from 50 to 600, in particular from 60 to 400 and especially from 75 to 200, and is more particularly about 100;

mixtures of polymers of formula (VIII) combining:

1) 80% to 99% by weight of a polymer of formula (VIII) in which m ranges from 50 to 600, in particular from 60 to 400 and especially from 75 to 200, and is more particularly about 100, and

2) 1% to 20% of a polymer of formula (VIII) in which m is in the range from 5 to 100 and in particular from 10 to 75, and is more particularly about 15;

mixtures of polymers of formula (VIII) combining:

1) 80% to 99% by weight of polymers of formula (VIII) in which n is equal to 2 to 10 and in particular from 3 to 6, and

2) 1% to 20% of polymers of formula (VIII) in which n is in the range from 5 to 500 and in particular from 30 to 100;

mixtures of polymers of formula (VIII) combining:

1) 1% to 20% of polymers of formula (VIII) in which n is equal to 2 to 10 and in particular from 3 to 6, and 2) 80% to 99% by weight of polymers of formula (VIII) in which n is in the range from 30 to 500 and in particular from 30 to 100;

polymers of formula (VIII) in which X represents a C₃ to C₁₅, in particular C₅ to C₁₂ and especially C₁₀ alkyl radical, and

polymers of formula (VIII) in which Y represents a C₃ to C₁₀, in particular C₄ to C₈ and especially C₆ alkyl radical.

As in the case of the polyamides, copolymers of polyurethane or polyurea silicone and of hydrocarbon-based polyurethane or polyurea may be used in the invention by performing the reaction for synthesizing the polymer in the presence of an α,ω-difunctional block of non-silicone nature, for example a polyester, a polyether or a polyolefin.

As has been seen previously, the copolymers of the invention may contain siloxane units in the main chain of the polymer and groups capable of establishing hydrogen interactions, either in the main chain of the polymer or at the ends thereof, or on side chains or branches of the main chain. This may correspond to the following five arrangements: 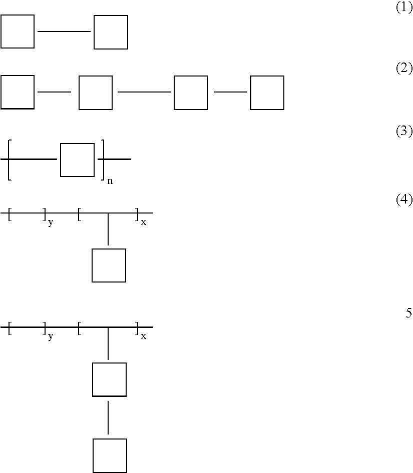
in which the continuous line is the main chain of the siloxane polymer and the squares represent the groups capable of establishing hydrogen interactions.

In case (1), the groups capable of establishing hydrogen interactions are located at the ends of the main chain. In case (2), two groups capable of establishing hydrogen interactions are located at each of the ends of the main chain.

In case (3), the groups capable of establishing hydrogen interactions are located within the main chain in repeating units.

In cases (4) and (5), these are copolymers in which the groups capable of establishing hydrogen interactions are located on branches of the main chain of a first series of units that are copolymerized with units not comprising groups capable of establishing hydrogen interactions. The values n, x and y are such that the polymer has the desired properties in terms of a structuring agent for fatty phases based on silicone oil.

According to one variant of the invention, the texture of the liquid fatty phase containing at least one oil is obtained with the aid of one or more of the polymers mentioned above.

As examples of polymers that may be used, mention may be made of the silicone polyamides obtained in accordance with Examples 1 to 3 of document U.S. Pat. No. 5 981 680.

They show good solubility in silicone oils and ester oils and lead to macroscopically homogeneous compositions. They preferably have an average molecular mass of from 500 to 200 000, for example from 10 000 to 150 000 and preferably from 20 000 to 100 000.

The silicone copolymer(s) may represent from 0.5% to 30% by weight, especially from 1% to 30% by weight and more particularly 5% to 30% by weight of the composition.

Non-Silicone Organic Copolymer

According to a second variant of the invention, the organic copolymer is a non-silicone copolymer.

More specifically, they are polymers and more particularly copolymers resulting from the copolymerization of at least one ethylenic monomer: vinyl, acrylic or methacrylic copolymers may more particularly be used. This compound may comprise, for example, a styrene (S) block or an alkylstyrene (AS) block, and a block chosen from ethylene/butylene (EB), ethylene/propylene (EP), butadiene (B), isoprene (I), acrylate (A) and methacrylate (MA) blocks, or a combination of these blocks.

In one particular embodiment, a copolymer comprising at least one styrene block is used. A triblock copolymer may be used and in particular those of the polystyrene/polyisoprene or polystyrene/polybutadiene type, such as those sold or manufactured under the name “Luvitol HSB” by BASF, and those of the polystyrene/copoly(ethylene-propylene) type or alternatively of the polystyrene/copoly(ethylene-butylene) type, such as those sold or manufactured under the brand name “Kraton” by Shell Chemical Co. or Gelled Permethyl 99A by Penreco. Styrene-methacrylate copolymers may also be used.

As ethylenic gelling agents that may be used in the compositions in accordance with the invention, examples that may be mentioned include Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS), Kraton D-1107 (SIS), Gelled Permethyl 99A -750, Gelled Permethyl 99A-753-58, Gelled Permethyl 99A-753-59, Versagel 5970 and Versagel 5960 from Penreco, and OS 129880, OS 129881 and OS 84383 from Lubrizol (styrene-methacrylate copolymer).

Diblock or triblock copolymers such as polystyrene-copoly(ethylene/propylene) or polystyrene-copoly(ethylene/butylene), such as those described in patent applications WO 98/38981 and U.S. 2002/0,055,562, are also included in the present invention.

Liquid Fatty Phase

The cosmetic compositions in accordance with the present invention comprise a liquid fatty phase based on at least one oil.

a. Silicone Oil

According to one variant of the invention, the liquid fatty phase comprises at least one volatile silicone oil.

The volatile silicone oil may be chosen from linear or cyclic silicone oils with a flash point of greater than or equal to 40° C. and advantageously greater than the softening point of the structuring polymer and/or a viscosity of less than 8 cSt, such as linear or cyclic polydimethylsiloxanes (PDMS) containing from 3 to 7 silicon atoms.

The flash point is the temperature at which a fuel catches fire on contact with a flame.

Examples of such oils that may be mentioned include the compounds cited in Table 1 below.

Advantageously, the volatile oil has a flash point of greater than 60° C.

The non-volatile silicone oils may be polydimethylsiloxanes, polyalkylmethylsiloxanes, dimethicone copolyols, alkylmethicone copolyols, cetyldimethicone, silicones containing alkyl glyceryl ether groups, silicones containing amine side groups and dilauroyltrimethylolpropane siloxysilicate. The alkyl groups of these oils especially contain from 2 to 24 carbon atoms.

The non-volatile silicone oils that may be used in the liquid fatty phase of the invention, which contains at least one ester oil, may in particular be non-volatile linear polydimethylsiloxanes (PDMS) that are liquid at room temperature; polydimethylsiloxanes comprising alkyl, alkoxy or phenyl groups, which are pendent and/or at the end of a silicone chain, these groups each containing from 2 to 24 carbon atoms; phenylsilicones, for instance phenyl trimethicones, phenyl dimethicones, phenyl trimethylsiloxy diphenylsiloxanes, diphenyl dimethicones, diphenyl methyldiphenyl trisiloxanes, 2-phenylethyl trimethylsiloxysilicates, fluorosilicones containing one or more group(s) that is (are) pendent or at the end of a chain, containing from 1 to 12 carbon atoms, all or some of the hydrogen atoms of which are replaced with fluorine atoms, and dimethiconols, and mixtures thereof.

The liquid fatty phase may comprise, besides the essential ester oil according to the invention, at least one volatile silicone oil and at least one volatile non-silicone oil.

For the purposes of the invention, a silicone or non-silicone volatile oil has a flash point preferably of from 40 to 135° C. or has no flash point. The volatile oils have at room temperature (25° C.) and atmospheric pressure (760 mmHg) a vapour pressure ranging from 0.02 mmHg to 300 mmHg (2.66 Pa to 40 000 Pa) and better still ranging from 0.1 to 90 mmHg (13 Pa to 12 000 Pa). The non-volatile oils thus correspond to a vapour pressure of less than 0.02 mmHg (2.66 Pa).

The silicone oils have a viscosity advantageously chosen in the range from 5 to 800 000 cSt, preferably from 10 to 500 000 cSt and better still from 10 to 5000 cSt at 25° C.

TABLE 1

| | Flash point | Viscosity |
|---|---|---|
| Compound | (° C.) | (cSt) |
| Octyl trimethicone | 93 | 1.2 |
| Hexyl trimethicone | 79 | 1.2 |
| Decamethyl cyclopentasiloxane (cyclo- | 72 | 4.2 |
| pentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF 96 A from Shin-Etsu | 94 | 6 |
| PDMS (polydimethylsiloxane) | 56 | 1.5 |
| DC 200 (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 cSt) from Dow Corning | 102 | 3 |

In other words, the volatile silicone oil(s) may be chosen, for example, from the group consisting of the compounds of Table 1, heptamethyloctyltrisiloxane and dodecamethylpentasiloxane, and mixtures thereof.

The volatile silicone oil may also be chosen from the group of fluorosilicone oils such as silicones containing alkyl and perfluoroalkyl groups, silicones containing oxyethylene/oxypropylene (OE/OP) side groups and perfluoro groups, silicones containing perfluoro side groups and glycerolated side groups, and perfluoroalkylmethylphenylsiloxanes, these oils having a vapour pressure of greater than or equal to 0.02 mmHg.

The liquid fatty phase advantageously contains at least 5% and for example from 10% to 90% by weight of silicone oil(s). The liquid fatty phase advantageously contains at least one silicone oil that advantageously has a viscosity of less than 1000 cSt and better still less than 100 cSt.

b. Ester Oil

According to one variant of the invention, at least one of the oils of the liquid fatty phase is an oil known as an “ester oil”, which is chosen from esters of monocarboxylic acids with monoalcohols and polyalcohols.

Advantageously, the said ester corresponds to the following formula:
R₁—CO—O—R₂
where

R₁ represents a linear or branched alkyl radical of 1 to 40 carbon atoms and preferably of 7 to 19 carbon atoms, optionally comprising one or more ethylenic double bonds, and optionally substituted,

R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted.

The term “optionally substituted” means that R₁ and/or R₂ can bear one or more substituents chosen, for example, from groups comprising one or more hetero atoms chosen from O, N and S, such as amino, amine, alkoxy and hydroxyl.

Preferably, the total number of carbon atoms of R₁+R₂ is ≧9.

The ester used according to the invention may be referred to as a “short” ester.

R₁ may represent the residue of a linear or, preferably, branched fatty acid, preferably a higher fatty acid, containing from 1 to 40 and even better from 7 to 19 carbon atoms, and R₂ may represent a linear or, preferably, branched hydrocarbon-based chain containing from 1 to 40, preferably from 3 to 30 and even better from 3 to 20 (19 to 28, 8 to 27, 7 to 26 C) carbon atoms. Once again, preferably the number of carbon atoms of R₁+R₂≧9.

Examples of groups R₁ are those derived from fatty acids chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters that may be used in the fatty phases of the compositions of the invention include purcellin oil (cetostearyl octanoate), isononyl isononanoate, isopropyl myristate, 2-ethylhexyl palmitate, 2-octyldodecyl stearate, 2-octyldodecyl erucate, isostearyl isostearate, and heptanoates, octanoates, decanoates or ricinoleates of alcohols or polyalcohols, for example of fatty alcohols.

Advantageously, the esters are chosen from the compounds of formula (I) above, in which R₁ represents an unsubstituted linear or branched alkyl group of 1 to 40 carbon atoms and preferably of 7 to 19 carbon atoms, optionally comprising one or more ethylenic double bonds, and R₂ represents an unsubstituted linear or branched alkyl group of 1 to 40 carbon atoms, preferably of 3 to 30 carbon atoms and even better of 3 to 20 carbon atoms, optionally comprising one or more ethylenic double bonds.

Preferably, R₁ is an unsubstituted branched alkyl group of 4 to 14 carbon atoms and preferably of 8 to 10 carbon atoms, and R₂ is an unsubstituted branched alkyl group of 5 to 15 carbon atoms and preferably of 9 to 11 carbon atoms. Preferably, in formula (I), R₁—CO— and R₂ have the same number of carbon atoms and are derived from the same radical, preferably an unsubstituted branched alkyl, for example isononyl, i.e. the ester oil molecule is advantageously symmetrical.

The ester oil will preferably be chosen from the following compounds:

isononyl isononanoate,

cetostearyl octanoate,

isopropyl myristate,

2-ethylhexyl palmitate,

2-octyldodecyl stearate,

2-octyldodecyl erucate,

isostearyl isostearate.

The ester that is preferred among all is isononyl isononanoate, which allows an optimum production of compositions with excellent transparency or translucency combined with excellent transfer-resistance and tack-free properties.

Advantageously, the liquid fatty phase comprises from 0.5% to 100% by weight, preferably from 1% to 80% by weight, more preferably from 2% to 50% by weight and better still from 2% to 40% by weight of ester oil(s).

More particularly, the fatty phase comprises at least isononyl isononanoate as ester oil.

c. Non-Silicone Oil

The liquid fatty phase of the compositions according to the invention may also contain one or more volatile or non-volatile non-silicone oils. The volatile non-silicone oils may be chosen from the group of volatile hydrocarbon-based oils, esters and ethers, such as volatile hydrocarbons, for instance isododecane and isohexadecane, and C₈-C₁₆ isoparaffins.

The volatile non-silicone oil may also be chosen from fluoro oils such as perfluoropolyethers, perfluoroalkanes, for instance perfluorodecalin, perfluoroadamantanes, perfluoroalkyl phosphate monoesters, diesters and triesters, and fluoro ester oils.

As examples of volatile non-silicone oils that may be used in the invention, mention may be made of the compounds in Table 2 below.

TABLE 2

| Compound | Flash point (° C.) |
|---|---|
| Isododecane | 43 |
| Isohexadecane | 102 |
| Propylene glycol n-butyl ether | 60 |
| Ethyl 3-ethoxypropionate | 58 |
| Propylene glycol methyl ether acetate | 46 |
| Isopar L (C₁₁-C₁₃ isoparaffin) | 62 |
| Isopar H (C₁₁-C₁₂ isoparaffin) | 56 |

When the fatty phase comprises a volatile oil, it advantageously represents from 3% to 89.4% and better still from 5% to 60%, for example from 5% to 20%, relative to the total weight of the composition.

The liquid fatty phase may also contain other non-silicone oils, for example polar oils such as:

hydrocarbon-based plant oils with a high content of triglycerides consisting of fatty acid esters of glycerol in which the fatty acids may have varied chain lengths, these chains possibly being linear or branched, and saturated or unsaturated; these oils are in particular wheatgerm oil, corn oil, sunflower oil, shea oil, castor oil, sweet almond oil, macadamia oil, apricot oil, soybean oil, rapeseed oil, cottonseed oil, alfalfa oil, poppy seed oil, pumpkin seed oil, sesame seed oil, marrow oil, avocado oil, hazelnut oil, grapeseed oil, blackcurrant seed oil, evening primrose oil, millet oil, barley oil, quinoa oil, olive oil, rye oil, safflower oil, candlenut oil, passion flower oil and musk rose oil; or caprylic/capric acid triglycerides such as those sold by the company Stearines Dubois or those sold under the names Miglyol 810, 812 and 818 by the company Dynamit Nobel;

synthetic esters containing from 10 to 40 carbon atoms;

C₈ to C₂₆ fatty alcohols, for instance oleyl alcohol and octyldodecanol;

fatty acids, for instance oleic acid, linoleic acid or linolenic acid; and

mixtures thereof.

The liquid fatty phase may also contain apolar oils such as linear or branched, volatile or non-volatile hydrocarbons or fluorocarbons of synthetic or mineral origin, for instance volatile liquid paraffins (such as isoparaffins or isododecane) or non-volatile liquid paraffins and derivatives thereof, petroleum jelly, polydecenes, hydrogenated polyisobutene such as parleam, and squalane, and mixtures thereof.

Thus, the invention may be implemented, for example, with the following various fatty phases:

1) a fatty phase consisting of a mixture of oils comprising at least one ester oil and at least one volatile silicone oil;

2) a fatty phase consisting of a mixture of oils comprising at least one ester oil and at least one non-silicone volatile oil;

3) a fatty phase consisting of a mixture of oils comprising at least one ester oil, at least one volatile non-silicone oil and optionally a volatile silicone oil;

4) a fatty phase consisting of a mixture of oils comprising at least one ester oil, a non-volatile non-silicone oil and optionally at least one volatile non-silicone oil; and

5) a fatty phase as defined in 1) to 4), free of ester oil.

Generally, the liquid fatty phase represents from 5% to 95% and better still from 20% to 75% of the total weight of the composition.

Other Fatty Substances

The compositions according to the invention may also comprise at least one solid fatty substance, which may be chosen from waxes and pasty compounds.

More particularly, the compositions according to the invention may comprise from 0.1% to 40% by weight, especially from 0.1% to 30% by weight and more particularly from 0.5% to 25% by weight of solid fatty substance(s) relative to the total weight of the composition.

For the purposes of the present invention, the term “wax” means a lipophilic compound that is solid at room temperature (25° C.), which undergoes a reversible solid/liquid change of state, and which has a melting point of greater than or equal to 30° C., which may be up to 120° C.

By bringing the wax to the liquid state (melting), it is possible to make it miscible with the oils that may be present and to form a microscopically homogeneous mixture, but on reducing the temperature of the mixture to room temperature, recrystallization of the wax in the oils of the mixture is takes place. The melting point of the wax may be measured using a differential scanning calorimeter (DSC), for example the calorimeter sold under the name DSC 30 by the company Mettler.

The wax may also have a hardness ranging from 0.05 MPa to 15 MPa and preferably ranging from 6 MPa to 15 MPa. The hardness is determined by measuring the compressive force, measured at 20° C. using the texturometer sold under the name TA-XT2i by the company Rheo, equipped with a stainless-steel cylinder 2 mm in diameter travelling at a measuring speed of 0.1 mm/s, and penetrating into the wax to a penetration depth of 0.3 mm.

The waxes may be hydrocarbon-based waxes, fluoro waxes and/or silicone waxes, and may be of plant, mineral, animal and/or synthetic origin. In particular, the waxes have a melting point of greater than 30° C. and better still greater than 45° C.

As waxes that may be used in the first composition of the invention, mention may be made of beeswax, carnauba wax or candelilla wax, paraffin, microcrystalline waxes, ceresin or ozokerite; synthetic waxes, for instance polyethylene waxes or Fischer-Tropsch waxes, and silicone waxes, for instance alkyl or alkoxy dimethicones containing from 16 to 45 carbon atoms.

The compositions may also contain a micronized wax, also known as a microwax.

As microwaxes that may be used in the compositions according to the invention, mention may be made of carnauba microwaxes, such as the product sold under the name “MicroCare 350®” by the company Micro Powders, synthetic microwaxes, such as that product sold under the name “MicroEase 114S®” by the company Micro Powders, microwaxes consisting of a mixture of carnauba wax and polyethylene wax, such as the products sold under the names “Micro Care 300®” and “310®” by the company Micro Powders, microwaxes consisting of a mixture of carnauba wax and of synthetic wax, such as the product sold under the name “Micro Care 325®” by the company Micro Powders, polyethylene microwaxes, such as the products sold under the names “Micropoly 200®” “220®”, “220L ®” and “250S®” by the company Micro Powders, and polytetrafluoroethylene microwaxes such as the products sold under the names “Microslip 519®” and “519 L®” by the company Micro Powders.

Among the microwaxes mentioned above, some of them, for instance carnauba microwax, the synthetic microwax “MicroEase 114S®” or the microwax consisting of a mixture of carnauba wax and of synthetic wax “MicroCare 325®”, have a starting melting point of greater than or equal to 45° C.

As a guide, the compositions may contain from 0.1% to 50% by weight and better still from 1% to 30% by weight of wax relative to their total weight.

These compositions may also contain at least one pasty compound, which may be chosen advantageously from:

lanolin and its derivatives

polymeric or non-polymeric silicone compounds

polymeric or non-polymeric fluoro compounds

vinyl polymers, especially:
olefin homopolymers
olefin copolymers
hydrogenated diene homopolymers and copolymers
linear or branched oligomers, homopolymers or copolymers of alkyl (meth)acrylates preferably containing a C₈-C₃₀ alkyl group
oligomers, homopolymers and copolymers of vinyl esters containing C₈-C₃₀ alkyl groups
oligomers, homopolymers and copolymers of vinyl ethers containing C₈-C₃₀ alkyl groups

liposoluble polyethers resulting from the polyetherification between one or more C2-C100 and preferably C₂-C₅₀ diols

esters, and

mixtures thereof.

Among the esters, the following are especially preferred:

esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, especially such as those sold under the brand name Softisan 649 by the company Sasol,

the arachidyl propionate sold under the brand name Waxenol 801 by Alzo,

phytosterol esters,

fatty acid triglycerides and derivatives thereof,

pentaerythritol esters,

non-crosslinked polyesters resulting from the polycondensation between a linear or branched C4-C50 dicarboxylic acid or polycarboxylic acid and a C2-C50 diol or polyol,

aliphatic esters of an ester, resulting from the esterification of an aliphatic hydroxy-carboxylic acid ester with an aliphatic carboxylic acid,

polyesters resulting from the esterification, with a polycarboxylic acid, of an aliphatic hydroxycarboxylic acid ester, the said ester comprising at least two hydroxyl groups, such as the products Risocast DA-H® and Risocast DA-L®, and

mixtures thereof.

Among the pasty compounds of plant origin that will preferably be chosen is a mixture of oxyethylenated (5 OE) oxypropylenated (5 OP) soybean sterols and of pentaerythritol, sold under the reference Lanolide by the company Vevy.

The composition may, where appropriate, comprise at least one aqueous phase, which may or may not consist essentially of water.

It may also comprise a mixture of water and of water-miscible organic solvent (water miscibility of greater than 50% by weight at 25° C.), for instance lower monoalcohols containing from 1 to 5 carbon atoms, such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes.

The aqueous phase (water and optionally the water-miscible organic solvent) may be present in a content ranging from 1% to 95% by weight, especially ranging from 3% to 80% by weight and in particular ranging from 5% to 60% by weight relative to the total weight of the composition.

Surfactant

The compositions according to the invention may also contain one or more surfactants.

According to one variant of the invention, they comprise at least one silicone surfactant.

The silicone surfactant(s) may be present in the composition in a content ranging from 0.1% to 50% by weight, in particular ranging from 0.1% to 40% by weight, more particularly ranging from 0.5% to 30% by weight, in particular ranging from 0.5% to 20% by weight, and even more particularly ranging from 1% to 10% by weight, relative to the total weight of the composition.

Among the surfactants that may be used in the cosmetic compositions in accordance with the present invention, mention may be made more particularly of hydrophilic organopolysiloxanes other than the silicone polymer described hereinabove.

The hydrophilic radical may correspond to the formula: [symbol]
CH₂ [symbol] ₚO [symbol] C₂H₄O)₁ [symbol] C₃H₆O [symbol] ᵣX
in which

p ranges from 0 to 5, q ranges from 0 to 100 and r ranges from 0 to 50, with p or q being non-zero,

the units (C₂H₄O) and (C₃H₆O) may be distributed randomly or in blocks, and

X is a hydrogen or a C₁-C₁₀ alkyl radical, where appropriate substituted with one or more functions of hydroxyl, thiol, amine, carboxylic, carboxylate, amide, phosphate, sulfate or sulfonate type.

In particular, p may range from 1 to 5, q from 1 to 100 and r from 1 to 50. X may more particularly feature a hydrogen atom.

In particular, the organopolysiloxane according to the invention may comprise as hydrophilic radical at least one hydroxy-polyalkylenoxy radical and especially a hydroxy-pblyethylenoxy radical.

The organopolysiloxane according to the invention may especially correspond to the formula: 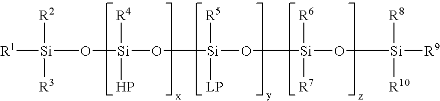
in which:

R¹, R², R³ , R⁴ , R⁵, R⁶₁ R⁷₁ R⁸ R⁹ and R¹⁰ represent, independently of each other, a linear, branched or cyclic, saturated or unsaturated C₁-C₆ alkyl radical,

HP is a radical bearing at least one hydrophilic group as defined hereinabove,

LP is a lipophilic radical, and

x ranges from 1 to 5000; y from 0 to 5000; z from 0 to 5000.

As regards the radical LP, it may be chosen especially from linear, branched or cyclic C₁-C₄₀ alkyls, organosiloxane groups, fluorine atoms and aryl, aryloxy, C₁-C₄₀ hydrocarbyl acyl and hydroxypropylenoxy radicals.

According to one particular variant of the invention, the organopolysiloxane belongs to the family of dimethicone-polyethylene glycols and may be chosen especially from the group comprising dimethicone copolyols, in particular cetyldimethicone copolyol and derivatives thereof. The hydrophilic organopolysiloxane according to the present invention may be the product sold under the brand name Abil WE 09 or Abil EM 90 by the company Degussa-Goldschmidt. The hydrophilic organopolysiloxane according to the present invention may also be the product sold under the reference KF-6017 by the company Shin-Etsu.

The organopolysiloxane compound may be totally or partially fluorinated. In particular, the lower dialkyl siloxy groups may be substituted with one or more fluorine atoms.

According to one particular embodiment, the silicone surfactant that may be used in the cosmetic compositions in accordance with the present invention is chosen from dimethicone copolyol, dimethicone copolyol benzoate, dimethicone copolyol phosphates, polyoxyalkylenated silicone elastomers and the cyclomethicone/dimethicone mixture, and mixtures thereof.

Polyoxyalkylenated silicone elastomers such as those described in patents U.S. Pat. No 5,236,986, U.S. Pat. No 5,412,004, U.S. Pat. No 5,837,793 and U.S. Pat. No 5,811,487, the content of which is incorporated by reference, are also suitable according to the invention.

Polyoxyalkylenated silicone elastomers that may be used include those sold under the names “KSG-21”, “KSG-20”, “KSG-30”, “KSG-31”, “KSG-32”, “KSG-33”, “KSG-210”, “KSG-310”, “KSG-320”, “KSG-330”, “KSG-340” and “X-226146” by the company Shin-Etsu, and “DC 9010” and “DC 9011” by the company Dow Corning.

It is understood that the compositions according to the invention may also comprise anionic and/or nonionic non-silicone surfactants.

For the choice of these surfactants, reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference, for the anionic and nonionic surfactants.

The surfactants that may be used more particularly in the composition according to the invention are chosen from:

nonionic surfactants: fatty acids, fatty alcohols, polyethoxylated or polyglycerolated fatty alcohols such as polyethoxylated stearyl or cetylstearyl alcohol, fatty acid esters of sucrose, alkyl glucose esters, in particular polyoxyethylenated fatty esters of a C₁-C₆ alkyl glucose, and mixtures thereof,

anionic surfactants: C₁₆-C₃₀ fatty acids neutralized with amines, ammonia or alkaline salts, and mixtures thereof.

Dyestuffs

According to one embodiment, the composition according to the invention may also contain at least one organic or mineral dyestuff, especially of the type such as pigments or nacres.

According to another embodiment, the composition according to the invention may also contain at least one dyestuff chosen from lipophilic dyes, hydrophilic dyes, pigments, nacres and materials with a specific optical effect, and mixtures thereof.

This dyestuff may be present in a proportion of from 0.01% to 50% by weight relative to the total weight of the composition, in particular from 0.5% to 40% by weight, more particularly from 5% to 25%, especially from 0.01% to 20% and in particular from 0.1% to 10%, or even from 2% to 5% by weight, relative to the total weight of the composition.

The term “pigments” should be understood as meaning white or coloured, mineral or organic particles, which are insoluble in an aqueous solution and which are intended to colour and/or opacify the resulting film.

The pigments may be present in a proportion of from 0.01% to 20% by weight, especially from 0.01% to 5% by weight and in particular from 0.02% to 7% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

They may also be pigments with a structure that may be, for example, of sericite/brown iron oxide/titanium dioxide/silica type. Such a pigment is sold, for example, under the reference Coverleaf® NS or JS by the company Chemicals and Catalysts, and has a contrast ratio in the region of 30.

The dyestuff may also comprise a pigment with a structure that may be, for example, of silica microsphere type containing iron oxide. An example of a pigment having this structure is the product sold by the company Miyoshi under the reference PC Ball® PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542 669, EP-A-787 730, EP-A-787 731 and WO-A-96/08537.

The term “nacres” should be understood as meaning iridescent or non-iridescent coloured particles of any form, especially produced by certain molluscs in their shell, or else synthesized, and which have a colour effect by optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the nacres available on the market, mention may be made of the nacres Timica®, Flamenco® and Duochrome® (on a mica base) sold by the company Engelhard, the Timiron® nacres sold by the company Merck, the Prestige® nacres on a mica base, sold by the company Eckart, and the Sunshine® nacres on a synthetic mica base, sold by the company Sun Chemical.

The nacres may more particularly have a yellow, pink, red, bronze, orange, brown and/or coppery colour or glint.

The pigments may or may not be surface-coated, in particular surface-treated with silicones, amino acids, fluoro derivatives or any other substance that promotes the dispersion and compatibility of the pigment in the composition.

Advantageously, the pigments used in the compositions in accordance with the invention may be surface-coated with a lecithin coating. This coating may be obtained by placing a solution of pigment in contact with a lecithin solution, in the presence of divalent or trivalent metal salts. Hydrogenated or non-hydrogenated lecithin may be used to obtain this coating.

The cosmetic composition according to the invention may also comprise water-soluble or liposoluble dyes in a content ranging from 0.01% to 10% by weight and especially ranging from 0.01% to 5% by weight relative to the total weight of the cosmetic composition.

The liposoluble dyes are, for example, Sudan red, DC Red 17, DC Green 6, β-carotene, soybean oil, Sudan brown, DC Yellow 11, DC Violet 2, DC Orange 5 and quinoline yellow.

When the cosmetic compositions according to the invention comprise a water-soluble dye, this dye may be present in the composition in dispersed form.

The cosmetic composition according to the invention may also contain at least one material with a specific optical effect.

This effect is different from a simple conventional hue effect, i.e. a unified and stabilized effect as produced by standard dyestuffs, for instance monochromatic pigments. For the purposes of the invention, the term “stabilized” means lacking an effect of variability of the colour as a function of the angle of observation or alternatively in response to a temperature change.

For example, this material may be chosen from particles with a metallic glint, goniochromatic colouring agents, diffracting pigments, thermochromic agents, optical brighteners, and also fibres, especially interference fibres. Needless to say, these various materials may be combined so as to simultaneously afford two effects, or even a novel effect in accordance with the invention.

The particles with a metallic glint that may be used in the invention are chosen in particular from:

particles of at least one metal and/or of at least one metal derivative,

particles comprising a mono-material or multi-material organic or mineral substrate, at least partially coated with at least one coat with a metallic glint comprising at least one metal and/or at least one metal derivative, and

mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr and mixtures or alloys thereof (for example bronzes and brasses) are preferred metals.

The term “metal derivatives” is intended to denote compounds derived from metals, especially oxides, fluorides, chlorides and sulfides.

As illustrations of these particles, mention may be made of aluminium particles, such as those sold under the names Starbrite 1200 EAC® by the company Siberline, and Metalure® by the company Eckart.

Mention may also be made of copper metal powders or alloy mixtures such as the reference 2844 sold by the company Radium Bronze, metallic pigments such as aluminium or bronze, such as those sold under the name Rotosafe® 700 from the company Eckart, the silica-coated aluminium particles sold under the name Visionaire Bright Silver® from the company Eckart and metal alloy particles, for instance the silica-coated bronze (alloy of copper and zinc) powders sold under the name Visionaire Bright Natural Gold® from the company Eckart.

They may also be particles comprising a glass substrate, such as those sold by the company Nippon Sheet Glass under the name Microglass Metashine®.

The goniochromatic colouring agent may be chosen, for example, from multilayer interference structures and liquid-crystal colouring agents.

Examples of symmetrical multilayer interference structures that may be used in the compositions prepared in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MOS₂/SiO₂/Al/SiO₂/MOS₂; Fe₂O₃/SiO₂/Al/SiO₂/Fe₂O₃, and Fe₂O₃/SiO₂/Fe₂O₃/SiO₂/Fe₂H₃, pigments having these structures being sold under the name Sicopearl by the company BASF; MOS₂/SiO₂/mica-oxide/SiO₂/MOS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of the Infinite Colors pigments from the company Shiseido. Depending on the thickness and the nature of the various layers, different effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO2 layers of 410 to 420 nm; from copper to red for SiO₂ layers of 430 to 440 nm.

Examples of pigments with a polymeric multilayer structure that may be mentioned include those sold by the company 3M under the name Color Glitter.

Examples of liquid-crystal goniochromatic particles that may be used include those sold by the company Chenix and also the products sold under the name Helicone® HC by the company Wacker.

Other Additives

The composition of the invention may also comprise any ingredient usually used in the field under consideration.

Needless to say, a person skilled in the art will take care to select the optional additional ingredients and/or the amount thereof such that the advantageous properties of the composition according to the invention are not, or are not substantially, adversely affected by the envisaged addition.

Thus, the composition according to the invention also comprises solid particles chosen from fillers.

The composition according to the invention may be in the form of a dermatological or care composition for keratin materials such as the skin, the lips and/or the integuments, in the form of an antisun composition or a makeup-removing composition, in stick form or in cast form. It may especially be used as a care base for the skin, the integuments or the lips (lip balms, for protecting the lips against the cold and/or the sun and/or the wind, or a care cream for the skin, the nails or the hair).

The composition of the invention may also be in the form of a coloured makeup product for the skin, in particular a foundation, optionally having care or treatment properties, a blusher, a makeup rouge, an eyeshadow, a concealer product, an eyeliner, a body makeup product; a lip makeup product, for instance a lipstick, a lip gloss or a lip pencil, optionally having care or treatment properties; a makeup product for the integuments, for instance the nails or the eyelashes, in particular in the form of a mascara cake, or for the eyebrows and the hair, especially in the form of a pencil. It may especially be in the form of an anhydrous stick.

Needless to say, the composition of the invention should be cosmetically or dermatologically acceptable, i.e. it should contain a non-toxic physiologically acceptable medium and should be able to be applied to human skin, integuments or lips. For the purposes of the invention, the term “cosmetically acceptable” means a composition of pleasant appearance, odour and feel.

The composition according to the invention may be manufactured via the known processes generally used in cosmetics or dermatology. It may be manufactured via the process that consists in heating the polymer at least to its softening point, adding the oil(s) thereto, and then mixing the whole. The dyestuffs, and/or the solid particles and the additives are then added, with stirring. The homogeneous mixture obtained may then be cast in a suitable mould, for instance a lipstick mould, or cast directly in the packaging articles (especially a case or dish).

According to another aspect, the invention also relates to a cosmetic assembly comprising:

i) a container delimiting at least one compartment, the said container being closed by means of a closing member; and

ii) a composition placed inside the said compartment, the composition being in accordance with the invention.

The container may be in any adequate form. It may especially be in the form of a bottle, a tube, a jar, a case, a box, a sachet or a carton.

The closing member may be in the form of a removable stopper, a lid, a cap, a tear-off strip or a capsule, especially of the type comprising a body attached to the container and a cover cap articulated on the body. It may also be in the form of a member for selectively closing the container, especially a pump, a valve or a flap valve.

The container may be combined with an applicator, especially in the form of a brush comprising an arrangement of bristles maintained by a twisted wire. Such a twisted brush is described especially in patent U.S. Pat. No 4,887,622. It may also be in the form of a comb comprising a plurality of application members, obtained especially by moulding. Such combs are described, for example, in patent FR 2 796 529. The applicator may be in the form of a fine brush, as described, for example, in patent FR 2 722 380. The applicator may be in the form of a block of foam or of elastomer, a felt or a spatula. The applicator may be free (tuft or sponge) or securely fastened to a rod borne by the closing member, as described, for example, in patent U.S. Pat. No 5,492,426. The applicator may be securely fastened to the container, as described, for example, in patent FR 2 761 959.

The product may be contained directly in the container, or indirectly. By way of example, the product may be arranged on an impregnated support, especially in the form of a wipe or a pad, and arranged (individually or in plurality) in a box or in a sachet. Such a support incorporating the product is described, for example, in patent application WO 01/03538.

The closing member may be coupled to the container by screwing. Alternatively, the coupling between the closing member and the container is done other than by screwing, especially via a bayonet mechanism, by click-fastening, gripping, welding, bonding or by magnetic attraction. The term “click-fastening” in particular means any system involving the crossing of a bead or cord of material by elastic deformation of a portion, especially of the closing member, followed by return to the elastically unconstrained position of the said portion after the crossing of the bead or cord.

The container may be at least partially made of thermoplastic material. Examples of thermoplastic materials that may be mentioned include polypropylene or polyethylene.

Alternatively, the container is made of non-thermoplastic material, especially glass or metal (or alloy).

The container may have rigid walls or deformable walls, especially in the form of a tube or a tubular bottle.

The container may comprise means for distributing or facilitating the distribution of the composition. By way of example, the container may have deformable walls so as to allow the composition to exit in response to a positive pressure inside the container, this positive pressure being caused by elastic (or non-elastic) squeezing of the walls of the container. Alternatively, especially when the product is in the form of a stick, the product may be driven out by a piston mechanism. Still in the case of a stick, especially of makeup product (lipstick, foundation, etc.), the container may comprise a mechanism, especially a rack mechanism, a threaded-rod mechanism or a helical groove mechanism, and may be capable of moving a stick in the direction of the said aperture. Such a mechanism is described, for example, in patent FR 2 806 273 or in patent FR 2 775 566. Such a mechanism for a liquid product is described in patent FR 2 727 609.

The container may consist of a carton with a base delimiting at least one housing containing the composition, and a lid, especially articulated on the base, and capable of at least partially covering the said base. Such a carton is described, for example, in patent application WO 03/018423 or in patent FR 2 791 042.

The container may be equipped with a drainer arranged in the region of the aperture of the container. Such a drainer makes it possible to wipe the applicator and possibly the rod to which it may be securely fastened. Such a drainer is described, for example, in patent FR 2 792 618.

The composition may be at atmospheric pressure inside the container (at room temperature) or pressurized, especially by means of a propellent gas (aerosol). In the latter case, the container is equipped with a valve (of the type used for aerosols).

The content of the patents or patent applications mentioned above are incorporated by reference into the present patent application.

The invention will now be described with reference to the examples that follow, which are given as non-limiting illustrations.

### EXAMPLE 1

Lipstick formulation

| INGREDIENTS | weight % |
|---|---|
| Polyamide/polydimethylsiloxane polymer | 25.00 |
| (n = 100) DC 2-8179 from Dow Corning | |
| Fragrance | 0.40 |
| Brown, yellow iron oxides | 1.00 |
| Black iron oxides | 0.47 |
| Titanium dioxide | 0.57 |
| Phenyl trimethicone | 28.00 |
| Red 7 | 1.10 |
| Isononyl isononanoate | 20.00 |
| Hydrogenated polyisobutene | 17.46 |
| Cetyl PEG/PPG-10/1 dimethicone | 3.00 |
| Polyethylene wax | 3.00 |

The formula under consideration tested according to the protocols described hereinabove has the following texture characteristics:

hardness of 74 g

elasticity of 98%

### EXAMPLE 2

Lipstick formulation

| INGREDIENTS | | weight % |
|---|---|---|
| Fragrance | Fragrance | 0.30 |
| Brown, yellow iron oxides (75/25) (CI: | Dye | 1.00 |
| 77491 + 77492) | | |
| Black iron oxides (CI: 77499) | Dye | 0.47 |
| Hydrogenated isoparaffin (6-8 mol of | Fatty | 18.10 |
| isobutylene | substance | |
| Phenyl trimethylsiloxy trisiloxane | Silicone | 37.73 |
| (viscosity: 20 cSt; MW: 372) | | |
| Calcium salt of lithol B red | Dye | 1.10 |
| Polydimethylsiloxane (viscosity: cSt) | Silicone | 11.53 |
| Oxyethylenated polymethylcetyl | Silicone | 3.00 |
| dimethyl methylsiloxane (20/75/5 - | | |
| viscosity: 3000 cSt) | | |
| Alumina/silica/trimethylolpropane- | Dye | 0.57 |
| treated rutile titanium oxide (CI: | | |
| 77891) | | |
| Polyamide/polydimethylsiloxane of | Silicone | 1.20 |
| Example 3 of U.S. Pat. No. 5 981 680 (n = | | |
| 15) | | |
| Polyamide/polydimethylsiloxane DC 2- | Silicone | 25.00 |
| 8179 from Dow Corning (n = 100) | | |

The formula under consideration tested according to the protocols described hereinabove has the following texture characteristics:

hardness of 108 g

elasticity of 97%

### EXAMPLE 3

Makeup product

| INGREDIENTS | Weight % |
|---|---|
| Styrene/ethylene-butylene/styrene triblock | 8.000 |
| copolymer (Kraton G-1650) | |
| Hydrogenated isoparaffin (6-8 mol of | 77.860 |
| isobutylene) (Polysinlane) | |
| Alumina/silica/trimethylolpropane-treated | 0.570 |
| rutile titanium oxide (CI: 77891) | |
| Calcium salt of lithol B red | 1.100 |
| Black iron oxide (CI: 77499) | 0.470 |
| Brown, yellow iron oxides (75/25) (CI: 77491 + | 1.000 |
| 77492) | |
| Hydrogenated isoparaffin (6-8 mol of | 8.000 |
| isobutylene) | |
| Polyethylene wax (MW: 500) | 3.000 |
| | 100.000 |

The formula under consideration tested according to the protocols described hereinabove has the following texture characteristics:

hardness of 30 g

elasticity of 99%

1. Solid cosmetic composition, characterized in that it comprises a liquid fatty phase textured with an effective amount of at least one organic copolymer, and in that it has a hardness ranging from 10 to 250 g and an elasticity of greater than 80%.

2. Solid cosmetic composition, characterized in that it comprises at least one dyestuff and a liquid fatty phase textured with an effective amount of at least one organic copolymer, and in that it has a hardness ranging from 10 to 250 g and an elasticity of greater than 80%.

3. Solid cosmetic composition, characterized in that it comprises a liquid fatty phase textured with an effective amount of at least two different organic copolymers, and in that it has a hardness ranging from 10 to 250 g and an elasticity of greater than 80%.

4. Solid cosmetic composition, characterized in that it comprises a liquid fatty phase comprising at least one silicone oil, the said liquid fatty phase being textured with an effective amount of at least one organic copolymer, and in that it has a hardness ranging from 10 to 250 g and an elasticity of greater than 80%.

5. Solid cosmetic composition, characterized in that it comprises at least one silicone surfactant and a liquid fatty phase textured with an effective amount of at least one organic copolymer, and in that it has a hardness ranging from 10 to 250 9 and an elasticity of greater than 80%.

6. Composition according to any one of the preceding claims, characterized in that the said copolymer is chosen from:
1) hybrid copolymers comprising at least two units capable of establishing hydrogen interactions, these two units being located in the chain of the copolymer, especially a polyorganosiloxane comprising at least two units capable of establishing hydrogen interactions, these two units being located in the chain of the copolymer, and/or
2) hybrid copolymers comprising at least two units capable of establishing hydrogen interactions, these two units being located on grafts or branches, especially a polyorganosiloxane comprising at least two units capable of establishing hydrogen interactions, these two units being located on grafts or branches.

7. Composition according to claim 6, characterized in that the said units capable of establishing hydrogen interactions are chosen from ester, amide, sulfonamide, carbamate, thiocarbamate, urea, urethane, thiourea, oxamido, guanidino and biguanidino groups, and combinations thereof.

8. Composition according to any one of the preceding claims, in which the said copolymer comprises at least one unit corresponding to the formula: 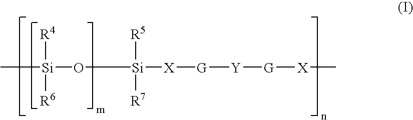
in which:
1) R⁴, R⁵, R⁶ and R⁷, which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms;
2) the groups X, which may be identical or different, represent a linear or branched C₁ to C₃₀ alkylenediyl group, possibly containing in its chain one or more oxygen and/or nitrogen atoms;
3) Y is a saturated or unsaturated, C₁ to C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups, or
4) Y represents a group corresponding to the formula: 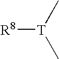
in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer;
5) the groups G, which may be identical or different, represent divalent groups chosen from: 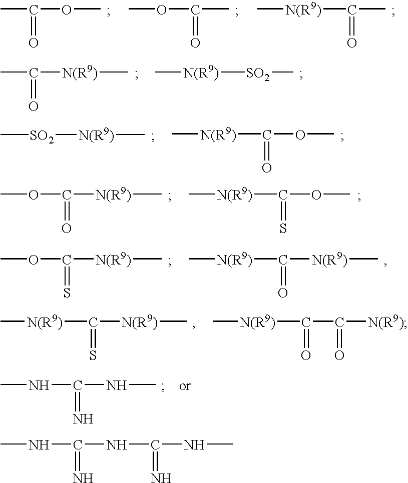
in which R⁹ represents a hydrogen atom or a linear or branched C₁ to C₂₀ alkyl group, on condition that at least 50% of the groups R⁹ of the polymer represent a hydrogen atom and that at least two of the groups G of the polymer are a group other than: 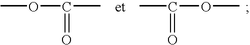
6) n is an integer ranging from 2 to 500 and preferably from 2 to 200, and m is an integer ranging from 1 to 1000, preferably from 1 to 700 and better still from 6 to 200.

9. Composition according to claim 8, in which Y represents a group chosen from:
a) linear C₁ to C₂₀ and preferably C₁ to C₁₀ alkylene groups,
b) C₃₀ to C₅₆ branched alkylene groups possibly comprising rings and non-conjugated unsaturations,
c) C₅-C₆ cycloalkylene groups,
d) phenylene groups optionally substituted with one or more C₁ to C₄₀ alkyl groups,
e) C₁ to C₂₀ alkylene groups comprising from 1 to 5 amide groups,
f) C₁ to C₂₀ alkylene groups comprising one or more substituents chosen from hydroxyl, C₃ to C₈ cyclo-alkane, C₁ to C₃ hydroxyalkyl and C₁ to C₆ alkylamine groups, and
g) polyorganosiloxane chains of formula: 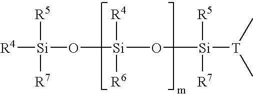
in which R⁴, R⁵, R⁶, R⁷, T and m are as defined in claim 8.

10. Composition according to any one of claims 1 to 9, in which the copolymer comprises at least one unit of formula (III) or (IV): 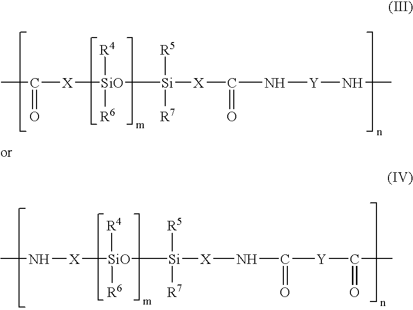
in which R⁴, R⁵, R⁶, R⁷, X, Y, m and n are as defined in claim 8.

11. Composition according to claim 10, in which X and/or Y represent(s) an alkylene group containing in its alkylene portion at least one of the following elements:
1) 1 to 5 amide, urea, urethane or carbamate groups,
2) a C₅ or C₆ cycloalkyl group, and
3) a phenylene group optionally substituted with 1 to 3 identical or different C₁ to C₃ alkyl groups and/or substituted with at least one element chosen from the group consisting of:
a hydroxyl group,
a C₃ to C₈ cycloalkyl group,
one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

12. Composition according to either of claims 10 and 11, in which Y represents: 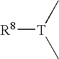
in which R⁸ represents a polyorganosiloxane chain and T represents a group of formula: 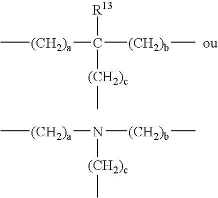
in which a, b and c are, independently, integers ranging from 1 to 10, and is a hydrogen atom or a group such as those defir or R⁴ R⁵, R⁶ and R⁷ in claim 8.

13. Composition according to any one of claims 10 to 12, in which R⁴, R⁵, R⁶ and R⁷ represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

14. Composition according to any one of claims 1 to 13, in which the copolymer comprises at least one unit of formula (VII): 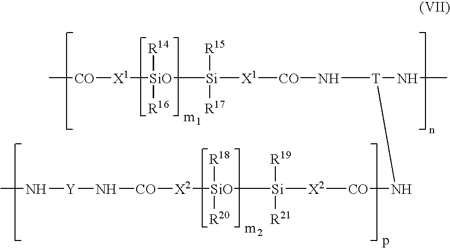
in which X¹ and X², which may be identical or different, have the meaning given for X in claim 8, n, Y and T are as defined in claim 8, R¹⁴ to R²¹ are groups chosen from the same group as R⁴ to R⁷ of claim 8, m₁ and m₂ are numbers in the range from 1 to 1000, and p is an integer ranging from 2 to 500.

15. Composition according to claim 14, in which:
p is in the range from 1 to 25 and better still from 1 to 7,
R¹⁴ to R²¹ are methyl groups,
T corresponds to one of the following formulae: 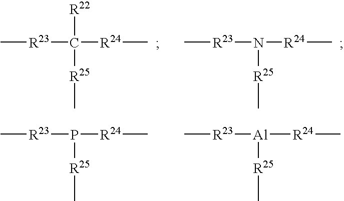
in which R²² is a hydrogen atom or a group chosen from the groups defined for R⁴ to R⁷, and R²³, R²⁴ and R²⁵ are, independently, linear or branched alkylene groups, and more preferably correspond to the formula: 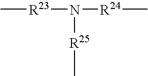
in particular with R²³, R²⁴ and R²⁵ representing —CH₂—CH₂—,
m₁ and m₂ are in the range from 15 to 500 and better still from 15 to 45,
X¹ and X² represent —(CH₂)₁₀—, and
Y represents —CH₂—.

16. Composition according to any one of claims 1 to 15, in which the copolymer comprises at least one unit corresponding to the following formula (VIII): 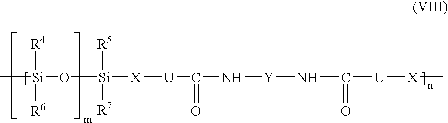
in which R⁴, R⁵, R⁶, R⁷I X, Y, m and n have the meanings given above for formula (I) in claim 8, and U represents —O— or —NH—, such that: 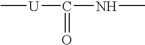
corresponds to a urethane or urea group, or
Y represents a C₅ to C₁₂ cycloaliphatic or aromatic group that may be substituted with a C₁ to C₁₅ alkyl group or a C₅ to C₁o aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane, or Y represents a linear or branched C₁ to C₄₀ alkylene radical or a C₄ to C₁₂ cycloalkylene radical, or
Y represents a polyurethane or polyurea block corresponding to the condensation of several diisocyanate molecules with one or more molecules of coupling agents of the diol or diamine type, corresponding to formula (IX): 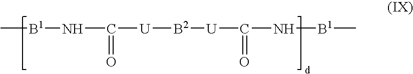
in which B¹ is a group chosen from the groups given above for Y, U is —O— or —NH— and B2 is chosen from:
linear or branched C₁ to C₄₀ alkylene groups,
C₅ to C₁₂ cycloalkylene groups, optionally bearing alkyl substituents, for example one to three methyl or ethyl groups, or alkylene, for example the diol radical: cyclohexanedimethanol,
phenylene groups that may optionally bear C₁ to C₃ alkyl substituents, and
groups of formula: 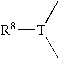
in which T is a hydrocarbon-based trivalent radical possibly containing one or more hetero atoms such as oxygen, sulfur and nitrogen and R⁸ is a polyorgano-siloxane chain or a linear or branched C₁ to C₅₀ alkyl chain.

17. Cosmetic composition according to any one of the preceding claims, characterized in that the copolymer is chosen from polymers comprising at least one unit corresponding to formula (II): 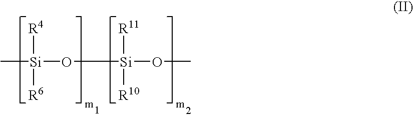
in which
R⁴ and R⁶₁ which may be identical or different, are as defined above for formula (I),
R¹⁰ represents a group as defined above for R⁴ and R⁶, or represents a group of formula —X-G-R¹² in which X and G are as defined above for formula (I) and R¹² represents a hydrogen atom or a linear, branched or cyclic, saturated or unsaturated, C₁ to CSO hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R¹¹ represents a group of formula —X-G-R¹² in which X, G and R¹² are as defined above,
m₁ is an integer ranging from 1 to 998, and
m₂ is an integer ranging from 2 to 500.

18. Composition according to any one of the preceding claims, characterized in that the texturing copolymer is chosen from:
polyamides of formula (III) as defined in claims 10 to 15, in which m ranges from 50 to 600, in particular from 60 to 400 and especially from 75 to 200, and is more particularly about 100;
mixtures of polyamide of formula (III) combining:
1) 80% to 99% by weight of a polyamide in which m ranges from 50 to 600, in particular from 60 to 400 and especially from 75 to 200, and is more particularly about 100, and
2) 1% to 20% of a polyamide in which m is in the range from 5 to 100 and in particular from 10 to 75, and is more particularly about 15;
mixtures of polyamide of formula (III) combining:
1) 80% to 99% by weight of a polyamide in which n ranges from 2 to 10 and in particular from 3 to 6, and
2) 1% to 20% of a polyamide in which n is in the range from 5 to 500 and in particular from 30 to 100;
mixtures of polyamide of formula (III) combining:
1) 1% to 20% by weight of a polyamide in which n is equal to 2 to 10 and in particular from 3 to 6, and
2) 80% to 99% of a polyamide in which n is in the range from 30 to 500 and in particular from 30 to 100;
polyamides of formula (III) in which X represents a C₃ to C₁₅, in particular C₅ to C₁₂ and especially C₁₀ alkyl radical, and
polyamides of formula (III) in which Y represents a C₃ to C₁₀, in particular C₄ to C₈ and especially C₆ alkyl radical.

19. Composition according to any one of the preceding claims, characterized in that it comprises at least one polydimethylsiloxane block copolymer of general formula I as defined according to any one of claims 8 to 16 and having an index m of greater than 50, in particular greater than 75 and especially of about 100.

20. Composition according to claim 19, characterized in that it also comprises at least one polydimethylsiloxane block copolymer of general formula I as defined according to any one of claims 8 to 16, having an index m of 15.

21. Solid cosmetic composition, characterized in that it comprises at least one liquid fatty phase textured with at least two copolymers of general formula I as defined according to any one of claims 8 to 16, 19 and 20, the two copolymers having indices m of different values, one being less than or equal to 50, and the other greater than or equal to 50.

22. Solid cosmetic composition according to the preceding claim, characterized in that it comprises:
1% to 90% by weight of at least one silicone oil,
5% to 50% by weight of copolymer(s) with an index m of greater than or equal to 50, and especially equal to 100,
0.1% to 10% by weight of copolymer(s) with an index m of less than or equal to 50, and especially equal to 15,
0.1% to 10% by weight of a silicone surfactant, and
0.001% to 40% by weight of at least one dyestuff.

23. Composition according to claim 21 or 22, characterized in that it has a hardness ranging from 10 to 250 g and an elasticity of greater than 80%.

24. Solid cosmetic composition for making up the lips, characterized in that it comprises at least one liquid fatty phase textured with an effective amount of at least one copolymer of general formula I as defined according to any one of claims 8 to 16, 19 and 20 and in which m is greater than or equal to 50.

25. Solid cosmetic composition, characterized in that it comprises at least one liquid fatty phase textured with an effective amount of at least one ethylenic copolymer, and in that it has a hardness ranging from 10 to 250 g and an elasticity of greater than 80%.

26. Composition according to claim 25, characterized in that the copolymer results from the copolymerization of an ethylenic monomer, in particular using vinyl, acrylic or methacrylic copolymers.

27. Composition according to claim 25 or 26, characterized in that the said copolymer comprises a styrene (S) block or an alkylstyrene (AS) block, and a block chosen from ethylene/butylene (EB), ethylene/propylene (EP), butadiene (B), isoprene (I), acrylate (A) and methacrylate (MA) blocks, or a combination of these blocks.

28. Composition according to claim 25, 26 or 27, characterized in that the said copolymer comprises at least one styrene block.

29. Composition according to any one of claims 25 to 28, characterized in that the said copolymer is chosen from block copolymers of polystyrene/polyisoprene, polystyrene/polybutadiene, polystyrene/copoly(ethylene-propylene), polystyrene/copoly(ethylene/butylene), styrene-methacrylate copolymer, polystyrene-copoly(ethylene/propylene) copolymer or polystyrene-copoly(ethylene/butylene) copolymer type.

30. Composition according to any one of the preceding claims, characterized in that its hardness ranges from 30 g to 200 g, and especially from 50 g to 170 g and more particularly from 75 g to 110 g.

31. Composition according to any one of the preceding claims, characterized in that its elasticity is greater than 90% and is especially close to 100%.

32. Composition according to any one of the preceding claims, characterized in that it comprises from 0.5% to 80% relative to the total weight of the composition, preferably from 2% to 60% and better still from 5% to 40% by weight of copolymer(s) relative to the total weight of the composition.

33. Composition according to any one of the preceding claims, characterized in that the texturing copolymer/liquid fatty phase mass ratio of the composition is from 0.01 to 10, especially from 0.05 to 5 and in particular from 0.1 to 2.

34. Composition according to any one of the preceding claims, characterized in that it comprises at least one silicone oil with a flash point of greater than or equal to 40° C. and advantageously greater than the softening point of the texturing polymer.

35. Composition according to claim 34, in which the silicone oil is chosen from the group consisting of the following compounds: octyl trimethicone, hexyl trimethicone, decamethylcyclo-pentasiloxane D5, octamethylcyclotetrasiloxane D4, dodecamethylcyclohexasiloxane D4, dodecamethyl-cyclohexasiloxane D6, heptamethyloctyltrisiloxane, decamethyltetrasiloxane, dodecamethylpentasiloxane, polydimethylsiloxane of 1.5 cSt, polydimethylsiloxane of 2 cSt, polymethylsiloxane of 3 cSt and polydimethylsiloxane of 5 cSt, and mixtures thereof.

36. Composition according to any one of claims 1 to 35, characterized in that it comprises at least one non-volatile silicone oil.

37. Composition according to any one of claims 1 to 36, characterized in that the liquid fatty phase contains at least 5% and in particular from 10% to 90% by weight of silicone oil(s).

38. Composition according to any one of the preceding claims, characterized in that it also comprises at least one ester oil chosen from esters of monocarboxylic acids with monoalcohols and polyalcohols.

39. Composition according to claim 38, characterized in that the said ester corresponds to the following formula:
R₁—CO—O—R₂
where
R₁ represents a linear or branched alkyl radical of 1 to 40 carbon atoms and preferably of 7 to 19 carbon atoms, optionally comprising one or more ethylenic double bonds, and optionally substituted,
R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted.

40. Composition according to claim 38 or 39, characterized in that the ester oil is chosen from the following compounds: isononyl isononanoate, cetostearyl octanoate, isopropyl myristate, 2-ethylhexyl palmitate, 2-octyldodecyl stearate, 2-octyldodecyl erucate, isostearyl isostearate, and heptanoates, octanoates, decanoates or ricinoleates of alcohols or polyalcohols.

41. Composition according to any one of the preceding claims, characterized in that the liquid fatty phase comprises from 0.5% to 100%, in particular from 1% to 80%, especially from 2% to 50% and more particularly from 2% to 40% by weight of ester oil(s).

42. Composition according to any one of the preceding claims, characterized in that it is in the form of an anhydrous stick.

43. Composition according to any one of claims 1 to 23 and 25 to 42, characterized in that it is in the form of a mascara cake, an eyeliner, a foundation, a lipstick, a blusher, a body makeup product, an eyeshadow, a makeup rouge or a concealer product.

44. Cosmetic process for caring for, making up or treating human keratin materials, comprising the application to the keratin materials of a cosmetic composition according to any one of the preceding claims.

45. Synthetic support on which is present, on all or part of its surface, at least one coat of a composition according to any one of claims 1 to 44.

46. Use of at least one organic copolymer in combination with at least one liquid fatty phase, for the preparation of a solid cosmetic composition with a hardness ranging from 10 to 250 g and an elasticity of greater than 80%.

47. Use according to claim 46, characterized in that the said copolymer is as defined in claims 6 to 29.

48. Use of a copolymer of general formula (I) as defined in claims 7 to 16 and 19 to 21, in which m has a value of greater than or equal to 50, in a lip makeup composition.

49. Cosmetic assembly comprising:
a) a container delimiting at least one compartment, the said container being closed by means of a closing member; and
b) a composition placed inside the said compartment, the composition being in accordance with any one of claims 1 to 43.

50. Cosmetic assembly according to claim 49, characterized in that the container is formed, at least partly, of at least one thermoplastic material.

51. Cosmetic assembly according to claim 49, characterized in that the container is formed, at least partly, of at least one non-thermoplastic material, especially of glass or metal.

52. Assembly according to any one of claims 49 to 51, characterized in that, in the closed position of the container, the closing member is screwed onto the container.

53. Assembly according to any one of claims 49 to 51, characterized in that, in the closed position of the container, the closing member is coupled to the container other than by screwing, especially by click-fastening, bonding or welding.

54. Assembly according to any one of claims 49 to 53, characterized in that the composition is substantially at atmospheric pressure inside the compartment.

55. Assembly according to any one of claims 49 to 53, characterized in that the composition is pressurized inside the container.
